(19) United States
(12) Patent Application Publication
(10) Pub. No.: US 2002/0142055 A1
(43) Pub. Date: Oct. 3, 2002

(54) Treatment of immune system-modulated disorders

(75) Inventors: Noel John De Souza, Aurangabad (IN); Ravindra D. Yeole, Aurangabad (IN); Rasendrakumar Jha, Aurangabad (IN); Sapna Bagchi, Aurangabad (IN)

Correspondence Address: LADAS & PARRY, 26 WEST 61ST STREET, NEW YORK, NY, 10023, US

(73) Assignee: WOCKHARDT LIMITED

(21) Appl. No.: 10/033,366

(22) Filed: Dec. 27, 2001

Related U.S. Application Data
(60) Provisional application No. 60/258,848, filed on Dec. 28, 2000.
(60) Provisional application No. 60/258,849, filed on Dec. 28, 2000.

(51) Int. Cl. A61K 35/78

(52) U.S. Cl. 424/762

(57) ABSTRACT

Immunomodulating pharmacologically and biologically active compositions containing a standardized extract of the plant Tinosporia cordifolia, a process for their preparation and their use in dosage forms for healthcare, nutrition and treatment of disorders modulated by the immune system are described. The process includes preparation of an extract from plant parts of Tinosporia cordifolia and standardization of the extract by phagocytosis and LC-MS single ion recording assays. Methods of treating immunomodulatory deficiencies in healthcare are also disclosed using the compositions.

### FIELD OF THE INVENTION

The present invention relates to a novel method of treatment of health conditions associated with alteration or modulation of immunity. The present invention also pertains to a standardized extract of the plant Tinospora cordifolia, compositions containing the extract and use of the extract to treat health conditions and for treatment of disorders modulated by the immune system.

### BACKGROUND OF THE INVENTION

The immune system of an organ acts as a defense mechanism regulated by an intricate system of humoral and cellular factors. Both humoral immune and cell-mediated immune mechanisms operate together on one hand to eliminate foreign bodies such as pathogenic microorganisms or neoplastic cells, and on the other to prevent the rejection of organ and tissue transplants. The immune system becomes deficient or compromised due to several reasons, namely genetic, debility, age, infections, cancer, auto immune mechanisms, and in recent years the acquisition of the immune deficiency syndrome (AIDS).

Immunocompromised conditions may be found in patients with the following infections, diseases, or disorders:

Ear, nose or throat (ENT) Infections: Chronic recurrent tonsillitis, Pharyngitis, Chronic otitis media, Peritonsillar abscess;

Respiratory system disorders: Tuberculosis, Chronic bronchitis, Chronic recurrent allergic bronchial asthma;

Gastrointestinal disorders: Recurrent Diarrhoea & Dysentery, Peritonitis, post-surgical abdominal infections;

Infections in immunocompromised host: Opportunistic infections in diabetes, Opportunistic infections in burns, opportunistic infections in malignancy;

Hepatobiliary diseases: Hepatitis, Cirrhosis of the liver, Obstructive jaundice;

Neutropenic patients: Patients on cancer chemotherapy;

Autoimmune diseases: destruction of pancreatic beta cells leading to Insulin Dependent Diabetic Mellitus (IDDM) and

Surgical prophylaxis.

Another disease related to immunity is osteomyelitis. Osteomyelitis is an infection of bone and is caused most commonly by pyogenic bacteria and mycobacteria. Microorganisms may enter bone in several ways, by the hematogenous route, by direct introduction from a contiguous focus of infection, or by a penetrating wound. They can bind to exposed sites of bone in which the susceptibility is enhanced by a variety of factors. The pathology of osteomyelitis is characterised by phenomena such as pus formation, lysed bone, devascularized bone fragments, subperiosteal or soft tissue abscesses, and in chronic cases, necrotic bone. Attendant symptoms are discharge, itching, odour, pain, tenderness and edema.

Especially in its chronic form, osteomyelitis is difficult to treat.

The treatment for osteomyelitis is based on classification of the disease, whether acute hematogenous, or vertebral, or secondary to a contiguous focus of infection, without or with vascular disease, and chronic forms of all such mentioned classes. Although current therapy reflects increased appreciation of the combined roles of antimicrobial courses and surgical debridement, the results especially in patients with chronic osteomyelitis are quite often discouraging.

Both approaches of antibiotic therapy and surgery are fraught with many limitations:

1) the usual need for initial intravenous administration of antibiotics; few data support the use of oral antibiotic by adults except in the case of fluoroquinolones; also the high dose of oral penicillins or cephalosporins recommended are not tolerated well by adults.

2) toxicity associated with the use of aminoglycosides like gentamicin and tobramycin, especially in cases of osteomyelitis due to Pseudomonas aeruginosa or Enterobacter sp.

3) prolonged courses of antimicrobial therapy, especially in chronic osteomyelitis.

4) multiple surgical procedures.

5) intraoperative difficulties to determine whether all necrotic and infected tissues are removed.

6) amputation or loss of an extremity.

Although immunological deficiencies in lymphocytic/macrophage cooperation and decrease in host defense cells CD5, CD4, CD8, natural killer cells and CD4/CD8 ratios have been shown to be implicated and associated in patients with chronic post-traumatic osteomyelitis, any definitive role in immune alteration is unclear in the etiology of chronic osteomyelitis (Peters K M, Klosterhalfen B, Zwadlo-Klarwasser G, Koberg K, Rosendahl T, Zilkens K W, Unfallchirurg 1993, 96(1):29-33; Sistermann R., Mollenhoff G, Walz M, Josten C, Muhr G, Unfallachirug 1993, 95(5):254-8). No immuno-adjuvant therapy is currently advocated in the clinical treatment of osteomyelitis.

Still another disease related to immunity is cancer. Cancer chemotherapy is associated with a fall in the number of circulating cells such as the red blood cells, the leukocytes and the platelets. Due to the property of cytotoxic drugs to kill non-malignant cells, the normal functional cells of the body are also destroyed. Thus because of a decrease, specifically in the leukocyte number, the patients who undergo chemotherapy are especially susceptible to fulminating infection during the course of therapy. Adjuvant therapies are needed to reduce the cytotoxic chemotherapy-induced leukopenia in cancer patients.

Another disease related to immunity is diabetes. Diabetes Mellitus is the most common endocrine disease found among human beings. It is characterized by hyperglycemia and glycosuria and in the long term it is associated with damage, dysfunction or failure of various organs, especially the eyes, kidneys, nerves, heart and blood vessels. Several lines of evidence suggest that insulin dependent diabetes mellitus (IDDM) results from autoimmune destruction of beta cells of the pancreas leading to insulin deficiency. Lymphocytic infiltrates indicating insulitis are seen during autopsies of patients with type-1 diabetes. Association of type-1 diabetes with polyendocrine autoimmunity and other autoimmune diseases also suggest this etiology. It is known that loss of insulin reserve occurs slowly over a few to many years and certain autoantibodies predate the development of the overt disease. One of the modes of therapy is initiating immunosuppressive therapy at the time of diagnosis of IDDM, which can prolong the patient's ability to secrete insulin, as determined by plasma C-peptide responses to a standard mixed meal or glucagon. This beneficial effect, whether achieved by Azathioprine, Cyclosporine or anti CD5 antibodies, is not sustained in most patients. The potential side effects of immunosuppressive agents, however, have precluded their use in large trials of non-diabetic subjects at increased risk of IDDM. Another interesting method of intervention involves orally induced tolerance to islet cell antigen implicated as targets of autoimmunity to beta cells. The beneficial effects of such immuno-modulatory therapy may result from the generation of T-lymphocytes that secrete cytokines (such as interleukin-4, interleukin-10, and transforming growth factor beta) which in turn retard the autoimmune responses to the subject's own myelin or collagen. A second therapy that may also generate regulatory cytokines capable of diminishing the destruction of beta cells is treatment with Bacille Calmette-Guerin. Various therapies have thus been tried along with conventional insulin therapy. However, their use has been limited because of minimum efficacy and the potential side effects.

Other diseases relate to respiratory system disorders. Chronic Obstructive Pulmonary Disease (COPD) is one of the common problems affecting 10% of population above the age of 45 years in the world. This is associated with frequent acute exacerbations and it contributes up to 25% of acute medical admissions to hospital. There is evidence to suggest that morbidity and mortality rates in COPD patients are rising and as such prompt and proper treatment of these patients is essential.

The specter of AIDS and the consequent alarming increase in the huge numbers of immune-compromised persons, and the high incidence of opportunistic infections have generated worldwide interest in the discovery of novel approaches and immunotherapy drugs to address the problems. So, too, is the case with drugs recommended for treatment of osteomyelitis, cancer, diabetes and respiratory system disorders. Many immunotherapy drugs and treatments have been found to be insufficiently effective and display toxic side effects. There is thus a need for newer, effective and safer approaches and drugs. Recourse is being had to alternate systems of medicine like Ayurveda to find herbal remedies that could provide immunoadjuvant therapies to conventional therapy that would raise the immune status of a patient to cope with the incurred disease.

Tinospora cordifolia (Menispermaceae), also known as guduchi, is named amrita in Ayurveda and has been used since ancient times for a variety of disorders. It belongs to the group of Rasayana and is found to be used in combination with other ayurvedic plants for the treatment of conditions associated with immunosuppression. In the prior art, laid-open application WO 91/08750 discloses the potential use of the cell contents of Tinospora cordifolia for the treatment of cancerous diseases. Only minimal clinical data is, however, provided for its use in cervix carcinoma. U.S. Pat. No. 5,529,778 describes an ayurvedic composition for the prophylaxis and treatment of AIDS, flu, tuberculosis and other immunodeficiency conditions. The composition comprises eight plant ingredients, one is a water extract of Tinospora cordifolia. The patent, however, discloses no way of preparing a standardized aqueous extract of Tinospora cordifolia, and discloses no indication whatever of the specific role or advantage, if any, of Tinospora cordifolia over the other plants in the composition. Indian Patent No. 183805, discloses a process for the preparation of an immunomodulator from the ayurvedic medicinal plant gulvel (Tinospora sp.), wherein the active principle is claimed to be a polysaccharide.

A product named Adbac is said to be commercially available in India as a natural immunostimulant in the form of capsules, reported to contain 300 mg of standardized aqueous extract of guduchi (Tinospora cordifolia). There is no indication in the published literature, however, of the manner by which the product is standardized.

A second product named Immumod is also known to be commercially available in India with indications for use in conditions associated with non specific suppression of immunity. Immumod is available as tablets of 100 mg/500 mg and as a syrup. Immumod is claimed to contain an aqueous extract of Tinospora cordifolia.

Standardized herbal products are the bane of the herbal health care industry. Herbal products are generally mixtures of several plants. Even when such products are of single plant constituents, there is usually no knowledge of the nature of the active ingredient(s) and of the amount required of the active ingredient in the extract for the product to be effective. Plant ingredients are known to vary depending on the strain of the plant used, the nature of the soil in which the plant grows, the age of the plant, the time of harvest and related factors. There is a great need, therefore, for herbal products to be standardized by methods that quantitate one or more of its ingredients to ensure that there is continuity of quality from one extract of the plant to another. Such a standardization enables treatment based on quantitative norms.

### SUMMARY OF THE INVENTION

The present invention relates to a novel method of treatment of health conditions associated with alteration or modulation of immunity. The present invention also pertains to a standardized extract of the plant Tinospora cordifolia, compositions containing the extract and use of the extract to treat health conditions and for treatment of disorders modulated by the immune system.

### BRIEF DESCRIPTION OF THE FIGURE

FIG. 1 illustrates the LC-MS SIR (Single Ion Recording) assay of a typical Tinospora cordifolia extract of the invention. The lowest trace depicts the fingerprint total ion chromatogram (TIC) of the methanol soluble portion of the extract. The middle trace showing one peak at 6.89 depicts the extracted mass chromatogram of selected ion (M+H)⁺ equal to 342 corresponding to the extract constituent of m/z 341 mass units. The upper trace showing one peak at 7.75 depicts the extracted mass chromatogram of selected ion (M+H)⁺ equal to 481 corresponding to the extract constituent of m/z 480 mass units.

### DETAILED DESCRIPTION OF THE INVENTION

The inventors of the present invention have conducted an extensive study to identify a therapy that can be used alone or in conjunction with other therapies. The inventors have found as a result that a herbal product, an extract of Tinospora cordifolia is suitable to be used alone or as an adjuvant therapy to other therapies such as antibiotic therapy, chemotherapeutic therapy and/or surgical therapy in effecting bacteriological, clinical and/or radiologic treatment in cases of health deficiencies associated with suppression of immunity, and in particular osteomyelitis, especially chronic osteomyelitis, cancer, diabetes and respiratory disorders. That is, the present invention relates in particular to an immunodulating agent, and its use in therapy alone or in adjuvant therapy.

The herbal-containing immunomodulatory agent according to the present invention is found by the present inventors to be effective as adjuvant therapy to conventional cancer chemotherapy in demonstrating the clinical efficacy by assessment of the decrease in the incidence of leukopenia in patients during cancer chemotherapy, especially breast cancer patients.

The herbal-containing immunomodulatory agent according to the present invention is also found by the present inventors to be effective as adjuvant therapy to conventional insulin therapy in demonstrating clinical efficacy by assessment of increase in the insulin secretory capacity, and a decrease in the daily insulin requirement. It is well tolerated.

The herbal-containing immunomodulatory agent according to the present invention is also found by the present inventors to be effective as adjuvant therapy to conventional antibiotic therapy and respiratory disorder amelioration therapy in demonstrating clinical efficacy in chronic bronchitis patients by assessment of the number of acute exacerbations, the forced expiratory volume and the peak expiratory flow.

The herbal-containing immunomodulatory agent according to the present invention is found to be effective for the treatment of osteomyelitis including the treatment of chronic osteomyelitis. Clinical efficacy is demonstrated by assessment of clinical parameters such as pain, tenderness, discharge, edema, itching, odor, in judging bacteriological cure as eradication or persistence of the initial causative pathogen in the post-treatment bacteriological examination, and in assessing radiological cure. It is well tolerated, causing few side effects.

The immunomodulating agent of the invention is a novel herbal extract prepared from the plant Tinospora cordifolia, which is standardized on the basis of its immunomodulatory activity as measured by its potential to increase phagocytosis by polymorphonuclear leukocytes (PMN) by a value of not less than 20% over a base value, and on the basis of its constituents, one of which has a mass spectrometric M+value of m/z 480 mass units and is present to an extent of not less than 35% of the two identified peak areas of the liquid chromatography mass spectrometry single ion recording (LC-MS-SIR) chromatogram, and the second of which has a mass spectrometric M+value of m/z 341 mass units and is present to an extent of not more than 65% of the two identified peak areas of the LC-MS-SIR chromatogram of the methanol soluble content of the extract. The process for the preparation of an extract of the plant Tinospora cordifolia comprises determining, by the technique of liquid chromatographymass spectrometry (LC-MS), and establishing a range within which the content in the extract must lie of one of its constituents having an M⁺value of m/z 480 mass units, and of a second of its constituents having an M⁺value of m/z 341 mass units, and determining and establishing a limit and range for a phagocytosis index within which the extract must lie. No extract of Tinospora cordifolia has been previously described which has been quantitatively standardized in the manner described herein.

The invention has become possible because of the in-depth studies of analysis of different extracts of Tinospora cordifolia that the inventors have conducted by the techniques of phagocytosis by polymorphonuclear (PMN) leukocytes, and of liquid chromatography spectrometry (LC-MS), and the identification of finger print patterns of immunomodulatory active extracts through these techniques. The use of the technique of phagocytosis by using PMN leukocytes as a measure of the immunomodulatory potential of the extract of the invention does not preclude or exclude the use of other methods to evaluate the immunomodulatory potential of the extract. Such methods are known to those skilled in the art and include the carbon clearance assay in rats (Wagner et al., Plant, Med., (3), 184, 1986), Jerne's spleen plaque assay (Science, 140, 405, 1963) or the uptake of tritiated thymidine by mouse spleen cells (Indian Patent No. 183805).

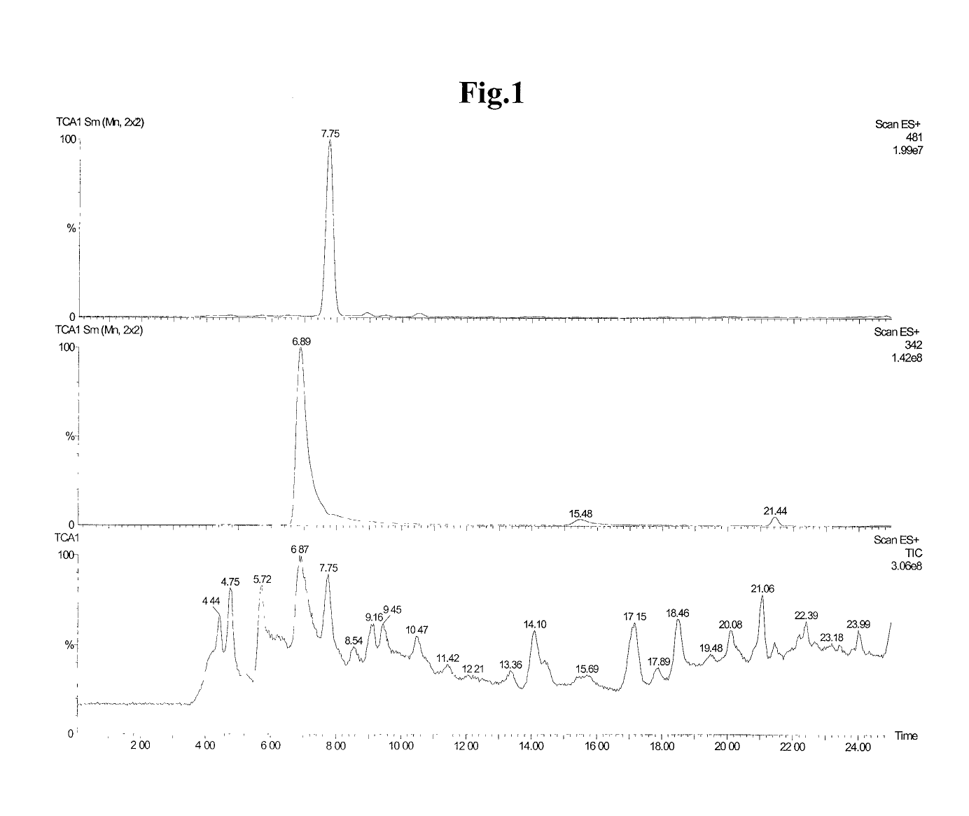

A number of extracts of Tinospora cordifolia of the invention prepared according to the standardized process of the invention as hereinbelow described was subjected to LC-MS assay. The details of the LC-MS assay method developed by the inventors is described in the experimental section. FIG. 1 displays in its lowest panel a typical total ion chromatogram (TIC) shown by the extracts of the invention.

The immunomodulatory activity of the extract is measured by determining a percentage increase in phagocytosis by PMN leukocytes over a base value according to the modified method of Lehrer (Lehrer et al., Blood 1968, 32, 423-35)—cf. Experimental section. All active extracts of the invention have a percentage increase of phagocytosis by PMN leukocytes of a value not less than 20% over a base value.

The stems and above-ground parts of the plant Tinospora cordifolia are used for preparation of the aqueous extract. The process for the preparation of an extract of Tinospora cordifolia comprises soaking the pulverized dried above-ground parts of the plant, Tinospora cordifolia with sufficient water to soak the plant material, raising the temperature to the boiling point, preferably by the passage of steam, for a period of about 1.5 hours to 2.5 hours, and separating the aqueous extract. The aqueous extract may be separated by draining, filtering, decanting or by any other method known in the art to separate aqueous parts of solutions or mixtures. Sufficient water to soak the residue is added and the steps of boiling and separating the aqueous extract are repeated. The steps of soaking, boiling and separating the aqueous extract is carried out for the third time. The aqueous extracts are pooled and are concentrated, preferably, under vacuum until the concentrate analyzes for a content of about 20% (w/v) of total solids. Preferably, the aqueous extract is concentrated under vacuum at temperatures of 50 to 60° C. The concentrate is cooled to room temperature and filtered. The filtrate is concentrated to a thick paste analyzing for a content of 60-70% (w/v) total solids. The thick paste is dried preferably in a vacuum drier at 50-60° C. until the dry material has a moisture content of less than 10% (w/v). In a preferred embodiment, the dried material is collected, pulverized in a mill, sieved over #20 sieve, and checked that it passes the pharmacopeal microbial limits. In the event the material does not meet the limits of the specification for microbial counts, the pulverized powder is treated with aqueous alcohol, preferably 50% aqueous alcohol, filtered and dried again, preferably in a vacuum drier, at 50-60° C. until the drug material has a moisture content of less than 10% (w/v) and assayed to ensure that it meets the pharmacopeal microbial limits.

The water used for the extraction of the plant material may be sterilized. The water is subjected to sterilization using one or more techniques known to those skilled in the art, viz. exposure to ultraviolet radiation, use of millipore filters, autoclaving, and preferably by exposure to UV light of wavelength 250-261 nm for varied periods of time dependent on the quality of the water.

The extract is evaluated for bioactivity by evaluating the percentage increase in phagocytosis by PMN leukocytes over a base value as described in the examples. An extract passes as an active extract when the percentage increase in phagocytosis is not less than 20% over a base valve. The methanol soluble portion of the extract is subjected to LC-MS assay. The quantitative range in which the peak M+value of m/z 480 mass units and the peak M+value of m/z 341 mass units lie is determined. An active extract displays a percentage increase in phagocytosis of not less than 20% over a base value, and contains a peak corresponding to M+value of m/z 480 mass units to an extent of not less than 35% of the two identified peak areas of the chromatogram, and also contains a second peak corresponding to M+value of m/z 341 mass units to an extent of not more than 65% of the two identified peak areas of the chromatogram of the methanol soluble content of the extract.

A further embodiment of the invention provides a pharmaceutical composition which comprises the standardized extract of Tinospora cordifolia of the invention and a pharmaceutically acceptable carrier, diluent, excipient or solvent.

The pharmaceutical compositions of this invention may be administered in standard manner for the disease or condition that it is desired to treat, for example by oral, topical, rectal or parenteral administration. For these purposes, the extracts of the invention may be formulated by means known in the art, for example as tablets, capsules, aqueous or oily solutions or suspensions, (lipid) emulsions, dispersable powders, suppositories, ointments, creams, eye drops, nasal drops and sterile injections and the like. Formulations known to those skilled in the art other than the above mentioned forms are also encompassed in the scope of this invention.

The extract is made available in the form of tablets, capsules and syrup, for oral administration. A suitable pharmaceutical composition of this invention is one suitable for oral administration. The dosage of the immunomodulating agent of the invention is appropriately selected according to the age, sex or other conditions or symptoms of the patient. A preferred dose of the agent is 1 to 50 mg/kg body weight in 3 or 4 divided doses per day preferably administered for a period of 5 to 7 weeks. Another dosage is a daily dose of from 25 mg to 1500 mg of the extract of Tinospora cordifolia. General recommendations for prescribing health care professionals is: a) for adults a tablet of 500 mg three times a day for a minimum of 15 days, b) for children a tablet of 100 mg three times a day for a minimum of 15 days and c) for children, aged 6 months to two years: ½ teaspoon 3 times daily; ages 7-12 years: 2 teaspoons 3 times daily or d) as directed by a physician.

In addition to the extract of the present invention the pharmaceutical composition of this invention may also contain, or be co-administered with, one or more known drugs selected from other clinically useful agents, in particular antibacterial agents, cancer chemotherapeutic agents, antidiabetic agents, and agents for treatment of bronchial diseases. The antibacterial agents may include penicillins, cephalosporins, fluoroquinolones, macrolides, carbapenems; the cancer chemotherapeutic agents may include cyclophosphamide, methotrexte, 5-fluorouracil; the antidiabetic agents may include insulin, and the agents for treatment of bronchial diseases may include theophylline and asthalin, all such agents being agents normally used in conventional therapy with which it is desired to have the immuno-adjuvant therapy of the invention done in conjunction. A suitable pharmaceutical composition of this invention is one suitable for oral administration in unit dosage form, for example, a tablet or capsule, which contains between 50 mg to 700 mg of the extract of the invention.

Yet another embodiment of the invention is the use of the standardized extract of Tinospora cordifolia of the invention and compositions thereof as adjuvant therapy in conjunction with other therapies for the treatment of different diseases due to immunodeficiency conditions. The product and compositions of the invention can be used for treatment of diseases such as osteomyelitis, chronic bronchitis, tuberculosis, lower respiratory tract infections, tonsillitis, otitis media, hepatitis, cancer, AIDS, diabetes mellitus, diabetic ulcers, bums and pediatric diseases.

A number of extracts of Tinospora cordifolia of the invention prepared according to the standardized process of the invention as hereinbelow described was subjected to LC-MS assay. The details of the LC-MS assay method developed by the inventors is described in the experimental section. FIG. 1 displays in its lowest panel a typical total ion chromatogram (TIC) shown by the extracts of the invention.

All types of cancer treated with chemotherapeutic agents or by radiation suppress the immune system and are thus conditions, which would be amenable to immunomodulating adjuvant therapy as is being advocated with the extract of the invention.

The extract of Tinospora cordifolia can also be used as a supplement in food and nutritional products. The invention is illustrated, but not limited by the following methods and examples.

### EXAMPLE 1

### Process for Making a Standardized Extract of Tinospora Cordifolia

Pulverized Tinospora cordifolia plant material (1 kg) is charged into a wooden vessel. UV sterilized water (2.5 lit. or sufficient quantity to soak the material) is added into the vessel and boiled with the help of steam (80° C.) for 2 hours. The aqueous extract is separated. Similar operation is repeated another two times. The collective extract is concentrated under vacuum to about 20% (w/v) of total solids, cooled to room temperature and filtered through 400 micron filter cloth in a filter press with the aid of supercell. The filtrate is concentrated to a thick paste of 60-70% (w/v) total solids. The thick paste is subjected to drying in a vacuum drier at 50-60° C. till the dry material has a moisture content less than 10% (w/v). The dry flakes collected are pulverized in a mill and sieved over 20 #.

### EXAMPLE 2

### LC-MS SIR Assay of Tinospora Cordifolia Extract

A number of extracts of Tinospora cordifolia of the invention prepared according to the standardized process of the invention were subjected to LC-MS assay. FIG. 1 displays in its lowest panel a typical total ion chromatogram (TIC) shown by the extracts of the invention.

### LC-MS SIR Assay

### Chemicals and Reagents

Ammonium acetate used was of analytical reagent grade. HPLC grade methanol, acetonitrile and double distilled water passed through Mill-Q water purification system were used throughout the experiment.

### Instrumentation

A Hewlett Packard HPLC (HP 1100) consisting of vacuum degasser, quaternary pump, autoinjector, thermostatted column compartment and variable wavelength UV detector was used. The chromatographic system consists of YMC-Pack-CN (250×4.6 mn, 5 microns, 120 Å) column and mobile phase (50 mM ammonium acetate and acetonitrile in gradient fashion) delivered at 1.0 ml/min. The thermostatted column compartment was maintained at 25° C. A gradient program was utilized ranging over 30 min with eluent percentages of acetonitrile increasing from 16% to 60% and reverting to 16%.

The autoinjector was set up to make 20 microliter injection with needle wash after each injection. The eluent from the column was split (3:1) using Valco splitter. The 75% eluent diverted to UV detector and 25% to electrospray probe of mass spectrometer. Mass spectrometric determination was performed on Micromass Quattro II, a triple quadrupole mass spectrometric operating in positive ion electrospray mode. The source temperature and desolvation temperature was 120° C. and 300° C. respectively. Nitrogen was used as drying gas and electrospray nebulising gas at the flow of 300 lit./hr. and 15 lit./hr. The ESI capillary potential was set at 4.0 kV and cone voltage was 30V. The LC-UV data was acquired at 240 nm. The LC-MS data was acquired from 150 to 700 Da with scan time of 1.3 sec and inter-scan delay 0.13 sec. Mass calibration and data acquisition were performed by using Windows NT based Masslynx 3.2 software. Peak areas of the UV chromatogram corresponding to m/z 341 mass units and to m/z 480 mass units were obtained by peak integration.

### Sample Preparation

Anhydrous extract powder (ca. 1 gm), prepared according to the process of the invention, was transferred to a 100 ml standard volumetric flask, and dissolved in methanol (100 ml) with the uses of sonication and shaking. About 50 ml was transferred to a centrifuge tube and centrifuged at 8000 rpm for 10 min. 20 ml of supernatant clear liquid was evaporated to dryness, 5 ml water was added to the residue, and the mixture was sonicated for 10 min. The mixture was filtered and passed through a previously conditioned SEP-PAK C-18 cartridge with 20 ml methanol followed by 20 ml water. The cartridge was rinsed with 10 ml water, and the retained components were eluted with 8 ml 50% aqueous methanol. The final volume was adjusted to 10 ml with aqueous fore the solution was used for LC-MS SIR assay.

### EXAMPLE 3

### Determination of Percentage Increase of Phagocytosis By PMN Leukocytes

The immunomodulatory activity of the extract is measured by determining a percentage increase in phagocytosis by PMN leukocytes over a base value. Polymorphonuclear (pmn) leukocytes phagocytosis assay was performed by a modified method of Lehrer (Lehrer et al., Blood 1968, 32, 423-35). Number of PMNs: 2×10⁶/ml; Test organisms (No.): Candida albicans (1×10⁶/ml); Concentration of test drug: 0.4 mg/ml.

TABLE 1
Percentage Increase In Phagocytosis By PMN
Leukocytes Over A Base Value

| Batch No. | % Increase in phagocytosis | | |
|---|---|---|---|
| of the | by PMN leukocytes over a | % peak area of | % peak area of |
| Invention | base value | M⁺ (m/z 480) | M⁺ (m/z 341) |
| 1 | 38.7 | 73.13 | 26.87 |
| 2 | 32.0 | 79.14 | 20.86 |
| 3 | 37.4 | 56.16 | 40.84 |
| 4 | 40.0 | 49.08 | 50.92 |
| 5 | 38.1 | 61.83 | 38.17 |

### EXAMPLE 4

The following illustrates representative pharmaceutical dosage forms containing the extract of the invention for therapeutic or prophylactic use in humans:

| (a) Tablet | |
|---|---|
| Tablet 1 | Mg/tablet |
| Extract of the invention | 55.00-700.00 |
| Microcrystalline cellulose | 10.00-127.00 |
| Lactose | 11.50-146.00 |
| Silicon dioxide | 2.00-25.40 |
| Cross carmellose sodium | 0.80-10.20 |
| Methyl paraben | 0.14-1.78 |
| Propyl paraben | 0.04-0.51 |
| Bronidiol | 0.02-0.25 |
| Magnesium stearate | 0.50-6.35 |
| For film coating of the tablets: | |
| Isopropyl alcohol | |
| Hydroxypropyl methyl cellulose | |
| Diethyl phthalate | |
| Methylene chloride | |
| Erythrocin aluminum lake | |
| Sunset yellow aluminum lake | |
| Ponceau 4 R aluminum lake | |
| Carnauba wax | |

(Aluminum lake is a water insoluble dye prepared from the dye and
aluminum oxide, and is used for coloring the tablets).

| | | |
|---|---|---|
| (b) Syrup | | |
| Syrup 1 | Qty/1.25 ml. | Qty/10 ml. |

| | | |
|---|---|---|
| Extract of the invention | 25.00 | 200.00 mg. |
| Sucrose | 0.63 | 5.00 gms. |
| Sodium methyl paraben | 1.88 | 15.00 mg. |
| Sodium propyl paraben | 0.63 | 5.00 mg. |
| Bronidiol | 0.25 | 2.00 mg. |
| Sodium saccharin | 2.50 | 20.00 mg. |
| Liquid glucose | 0.33 | 2.86 gms |
| Caramel | 1.20 | 10.00 ml. |
| Flavour cardamom 21180 | 0.0013 | 0.01 ml. |
| Purified water q.s. to | 1.25 | 10.00 ml. |

The above formulations may be obtained by conventional procedures well known in the pharmaceutical art. The tablet (a) may be film coated and a suitable color included by conventional means.

### EXAMPLE 5

Effect of the extract of the invention as adjuvant therapy in patients with osteomyelitis.

Extract of the invention (1 tablet, 500 mg) and matching Placebo tablets were administered twice daily for 6 weeks to a randomized group of 50 patients (36 males and 14 females) diagnosed as suffering from subacute to chronic osteomyelitis. In addition to the immunodulating extract of the invention, all patients received antibiotic therapy in the form of Tab pefloxacin (400 mg) twice daily for 6 weeks.

After 6 weeks from the start of the treatment with the tablets, the physician in charge judged the degree of symptomatic improvement, clinical efficacy, bacteriological response and radiological assessment in the patients on the immunomodulating extract of the invention/placebo therapy based on 3 scales of cure, improvement and failure. These results are shown in Table 2.

TABLE 2
Symptom Score/Cure Improvement Index
(I/P × 100)*

| | |
|---|---|
| Symptom Evaluation | 103 |
| Clinical Evaluation | 120 |
| Bacteriological Response | 119 |
| Radiological Assessment | 97 |

*I = Extract of the invention data
P = Placebo data

The results indicate the improvements seen with the immunomodulating agent in symptom evaluation, clinical evaluation and bacteriological response. Radiological improvements are known to be seen long after the drug treatment of an infective condition is completed.

It is seen from these results that the immunomodulating agent according to the present invention brings about improvement in symptoms and cure of osteomyelitis, especially chronic osteomyelitis, exhibiting effectiveness over patients treated only with conventional therapy.

### EXAMPLE 6

Effect of extract of the invention against cytotoxic chemotherapy induced leukopenia in breast cancer patients.

The extract of the invention (1 tablet 500 mg) and matching Placebo tablets were administered thrice daily for 14 days as per a chemotherapy cycle protocol to a randomized, double blind placebo clinical trial group of 38 patients diagnosed as suffering from breast cancer. All patients also received chemotherapy in the form of cyclophosphamide 750 mg/m², methotrexate 40 mg/m² and 5-flurouracil 750 mg/m² every 3 weeks. An absolute end point for each cycle of chemotherapy for every patient was the appearance of leukopenia (leucocyte, WBC count <3000 mm³). The results may be summarised as follows:

1. There was no difference in the basal WBC counts of both groups indicating that the groups were similar at the beginning.

2. There was a significant leukopenia in both the groups. However, the number of patients with total WBC counts less than 3000/cu mm were significantly less (p <0.05, chi square test) in the group administered the extract of the invention (55%) as compared to the placebo treated group (70%).

3. There were 24 cycles in the placebo group where the count fell below 2000/cu mm while there were only 14 in the treated group.

The results indicate that treatment with the immunomodulating extract of the invention was found to decrease the incidence of leukopenia in patients, especially in breast cancer patients, on cancer chemotherapy, exhibiting such effectiveness over patients treated with only conventional chemotherapy. The conclusion suggested is that the extract of the invention (a) induces leukocytosis thus increasing the WBC counts and (b) induces the release of granulocyte macrophage—colony stimulating factor (GM-CSF), as shown in animal studies, thus abating leukopenia.

### EXAMPLE 7

### Effect of The Extract of the Invention on the Insulin Secretory Reserve in Type 1 Diabetic Patients on Insulin Therapy

The extract of the invention (1 tablet, 500 mg) and matching Placebo tablets were administered thrice daily for 4 weeks to a randomized group of 50 patients (34 males and 16 females) diagnosed as suffering from IDDM. The patients in the group administered the extract of the invention as well as insulin therapy were designated as cases. The patients in the other group who received only insulin therapy were designated as controls. After 4 weeks from the start of the treatment with the tablets, the physician in charge judged the degree of clinical efficacy on the basis of the following parameters:

1. Blood glucose levels (Fasting and 2-hour post-load glucose)

2. Serum C-peptide levels basal, stimulated and percentage rise in the C-peptide level over the basal level 2-hours following 75 grams oral glucose load.

3. Glycosylated haemoglobin

4. Total daily insulin requirements

5. Quality of life/performance status

The results of the study in respect of the insulin secretory reserve and the daily insulin requirements were as follows:

1. The mean percentage rise in the insulin secretory reserve in the cases and the controls were 25.96±15.07 and 0.19±14.55 respectively which is highly statistically significant.

2. The mean percentage rise in the daily insulin requirements in cases was 20.17±26.59 and that in controls was 84.86±36.04 which is highly statistically significant.

The results indicate the improvements seen with the immuno-modulating agent in increased insulin secretory capacity and reduction in insulin requirement. It is seen from these results that the immunomodulating agent according to the present invention brings about improvement in symptoms and cure of IDDM exhibiting effectiveness over patients treated only with conventional therapy.

### EXAMPLE 8

### Effect of the Invention in Chronic Bronchitis Patients

The extract of the invention (1 tablet, 500 mg) and matching Placebo tablets were administered thrice daily for 8 weeks to a randomized group of 60 patients. In addition to the immunomodulating extract of the invention, all acute exacerbations were treated with Roxithromycin 150 mg BD, Theophylline 200 mg BD and asthalin rotahaler.

After 8 weeks of therapy, the physician in charge judged the degree of efficacy on the basis of the following parameters:

1. Reduction in number of Acute Exacerbations during 8 weeks of study compared to the previous 2 months.

2. Improvement in Forced Expiratory volume in 1 sec measured by spirometry.

3. Improvement in Peak expiratory flow measured by spirometry.

The results may be summarized s follows:

1. Significant reduction in the episodes of Acute Exacerbations in the test group 2.06±0.41 as compared to the control group 3.90±0.76.

2. Significant improvement in the Forced Expiratory volume in the test group 42.36±10.32 as compared to control group 33.63±5.73.

3. Significant improvement in the Peak Expiratory flow in the test group 30.70±8.37 as compared to control group 24.53±4.58.

The results indicate that the treatment with the immunomodulating extract of the invention was found to decrease the incidence of acute exacerbations in chronic bronchitis patients and to improve the forced expiratory volume and peak expiratory flow exhibiting such effectiveness over patients treated with only conventional therapy. It can be concluded that by inducing phagocytosis and release of GM-CSF, as shown in animal studies it decreases the incidence of infections and acute exacerbation in patients with chronic bronchitis.

Similarly data can be provided for treatment of animals in pharmacological models of immunomodulatory conditions and for treatment of humans suffering from disorders and diseases such as tuberculosis, lower respiratory tract infections, chronic obstructive pulmonary disorders, tonsilitis, otitis media, hepatitis, cancer, AIDS, diabetes mellitus, diabetic ulcers, bums and pediatric diseases.

1. A method of treatment of a health condition associated with modulation of immunity which method comprises administering a standardized herbal extract prepared from the plant Tinospora cordifolia.

2. A method of treatment of a health condition associated with modulation of immunity which method comprises administering a standardized herbal extract prepared from the plant Tinospora cordifolia in conjunction with another treatment for the health condition.

3. A method of treatment according to claim 1, in which the herbal extract is standardized on the basis of its immunomodulatory activity as measured by its potential to increase phagocytosis by polymorphonuclear leukocytes by a value of not less than 20% over a base value, and on the basis of its constituents, one of which has a mass spectrometric M+value of m/z 480 mass units and is present to an extent of not less than 35% of the two identified peak areas of the liquid chromatography mass spectrometry single ion recording (LC-MS SIR) chromatogram, and the second of which has a mass spectrometric M+value of m/z 341 mass units and is present to an extent of not more than 65% of the two identified peak areas of the LC-MS SIR chromatogram of the methanol soluble content of said extract.

4. A method of treatment of a health condition associated with modulation of immunity according to claim 2, in which the herbal extract is standardized on the basis of its immunomodulatory activity as measured by its potential to increase phagocytosis by polymorphonuclear leukocytes by a value of not less than 20% over a base value, and on the basis of its constituents, one of which has a mass spectrometric M+value of m/z 480 mass units and is present to an extent of not less than 35% of the two identified peak areas of the liquid chromatography mass spectrometry single ion recording (LC-MS SIR) chromatogram, and the second of which has a mass spectrometric M+value of m/z 341 mass units and is present to an extent of nor more than 65% of the two identified peak areas of the LC-MS SIR chromatogram of the methanol soluble content of said extract.

5. A method of treatment of a health condition associated with alteration or modulation of immunity which method comprises administering a standardized herbal extract prepared from the plant Tinospora cordifolia wherein the herbal extract is standardized on the basis of its immunomodulatory activity as measured by its potential to increase phagocytosis by polymorphonuclear leukocytes by a value of not less than 20% over a base value, and on the basis of its constituents, one of which has a mass spectrometric M+value of m/z 480 mass units and is present to an extent of not less than 35% of the two identified peak areas of the liquid chromatography mass spectrometry single ion recording (LC-MS SIR) chromatogram, and the second of which has a mass spectrometric M+value of m/z 341 mass units and is present to an extent of not more than 65% of the two identified peak areas of the LC-MS SIR chromatogram of the methanol soluble content of said extract in conjunction with another therapy for the health condition.

6. The method of treatment according to claim 2, wherein the condition is osteomyelitis.

7. The method of treatment according to claim 4, wherein the condition is osteomyelitis.

8. The method of treatment according to claim 5, wherein the condition is osteomyelitis.

9. The method of treatment according to claim 2, wherein the condition is cancer.

10. The method of treatment according to claim 4, wherein the condition is cancer.

11. The method of treatment according to claim 5, wherein the condition is cancer.

12. The method of treatment according to claim 2, wherein the condition is breast cancer.

13. The method of treatment according to claim 4, wherein the condition is breast cancer.

14. The method of treatment according to claim 5, wherein the condition is breast cancer.

15. The method of treatment according to claim 2, wherein the condition is type 1 diabetes.

16. The method of treatment according to claim 4, wherein the condition is type 1 diabetes.

17. The method of treatment according to claim 5, wherein the condition is type 1 diabetes.

18. The method of treatment according to claim 2, wherein the condition is tuberculosis, lower respiratory tract infections, tonsilitis, otitis media, hepatitis, cancer, AIDS, diabetes mellitus, diabetic ulcers, bums or pediatric disease.

19. The method of treatment according to claim 4, wherein the condition is tuberculosis, lower respiratory tract infections, tonsilitis, otitis media, hepatitis, cancer, AIDS, diabetes mellitus, diabetic ulcers, bums or pediatric disease.

20. The method of treatment according to claim 5 wherein the condition is tuberculosis, lower respiratory tract infections, tonsilitis, otitis media, hepatitis, cancer, AIDS, diabetes mellitus, diabetic ulcers, bums or pediatric disease.

21. The method according to claim 2, wherein the condition is a respiratory tract disease.

22. The method according to claim 4, wherein the condition is a respiratory tract disease.

23. The method according to claim 5, wherein the condition is a respiratory tract disease.

24. The method according to claim 2, wherein the condition is chronic bronchitis.

25. The method according to claim 4, wherein the condition is chronic bronchitis.

26. The method according to claim 5, wherein the condition is chronic bronchitis.

27. The method according to according to any one of claims 1 to 26, wherein the daily dosage is 1-50 mg/kg of body weight.

28. The method according to any one of claims 1 to 26, wherein the daily dosage is from 25 mg to 1500 mg.

29. A process for preparation of the standardized extract of Tinospora cordifolia which comprises treating the plant material with water at an elevated temperature, filtering and concentrating to provide an extract that meets the defined standardization limits as its immunomodulatory activity as measured by its potential to increase phagocytosis by polymorphonuclear leukocytes by a value of not less than 20% over a base value, and has one constituent which has a mass spectrometric M+value of m/z 480 mass units and is present to an extent of not less than 35% of the two identified peak areas of the liquid chromatography mass spectrometry single ion recording (LC-MS SIR) chromatogram, and has a second constituent which has a mass spectrometric M+value of m/z 341 mass units and is present to an extent of not more than 65% of the two identified peak areas of the LC-MS SIR chromatogram of the methanol soluble content of said extract.

30. An extract of Tinosporia cordifolia prepared by the process comprising treating the pulverized above ground parts of the plant Tinosporia cordifolia with water at an elevated temperature, filtering and concentrating to provide an extract that has immunomodulatory activity as measured by its potential to increase phagocytosis by polymorphonuclear leukocyte by a value of not less than 20% over a base value, and has one constituent which has a mass spectrometric M+value of m/z 480 mass units and is preset to an extent of not less than 35% of the two identified peak areas of the liquid chromatography mass spectrometry single ion recording (LC-MS SIR) chromatogram, and has a second constituent which has a mass spectrometric M+value of m/z 341 mass units and is present to an extent of not more than 65% of the two identified peak areas of the LC-MS SIR chromatogram of the methanol soluble content of said extract.

31. A composition comprising the extract of claim 30.
